(19) United States
(12) Patent Application Publication
(10) Pub. No.: US 2012/0259246 A1
(43) Pub. Date: Oct. 11, 2012

(54) REFLEX MEASURING APPARATUS DETERMINING THE REACTIVITY OF A DRIVER AND ITS APPLICATION

(75) Inventors: Gian Mario RUBBOLI, Milano (IT); Giorgio MARCON, Giavera Del Montello (IT)

(73) Assignee: ERBICOL SA, Balerna (CH)

(21) Appl. No.: 13/232,556

(22) Filed: Sep. 14, 2011

(30) Foreign Application Priority Data
Sep. 15, 2010 (CH) 01500/10

(51) Int. Cl. A61B 5/16

(52) U.S. Cl. 600/587

(57) ABSTRACT

Reflex measuring apparatus for determining the reactivity of a driver, or respectively, his ability to drive a vehicle under conditions including under the influence of alcohol or drugs, tiredness, stress or normal effects of age. Reflex measuring apparatus includes s a steering wheel of a vehicle that acts as a sensor for measuring the reaction time span of the driver, a signaling device soliciting the reaction of the driver, a device to record a time span between the solicitation by the signaling device and a reaction of the driver (e.g. an analogue or digital chronograph), and a seat provided with a safety belt. The apparatus can be activated when a driver takes place on the seat.

The present application claims priority under 35 U.S.C. §119(a) of Swiss Patent Application No. 01500/10 filed Sep. 15, 2010, the disclosure of which is expressly incorporated by reference herein in its entirety.

The present invention concerns a reflex measuring apparatus as described in the introductory part of the patent claim 1 and its practical application as provided in the claims 11 and 12.

The problem of determining the reactivity of a driver of a vehicle, i.e. the speed and reliability of reactions to the unexpected emergence of perils, represents one of the most important aspects of automobile accident prevention. Innumerable attempts have been undertaken over the past decades to reduce the number and the seriousness of road incidents, and have achieved measures related to the vehicles themselves (such as impact dampening arrangements, higher resistance of the vehicle structure and thus controlled deformability thereof in case of collision), as well as provisions aimed at improving the reactivity of the drivers in case of unexpected incidents, etc. Today in most countries severe laws are in force limiting blood alcohol contents to very low levels, measurable using various instruments and analysis methods. It is known, however, that the blood alcohol level is just one of many parameters determining the reactivity of a person. Tiredness, drug consumption, age and other psychic impairments strongly influence the effective driving capacity—manifest essentially in the speed of the reactions to unforeseen events—of the driver. The simple test for blood alcohol level alone thus does not represent a reliable method for determining the aptitude of a person of driving an automobile. Much safer, on the other hand, is a test measuring the reactivity of the driver, and this is the objective aimed at using the reflex measuring apparatus according to the present invention. In many countries similar tests are required by law for seniors, but as a rule such tests are simple checks of vision and hearing, which can not easily be considered tests of reactivity.

Decisive in this context was the development of a steering wheel for vehicles provided with a device reacting to the variations in pressure exerted by one or both hands of the driver gripping the rim of the steering wheel. As the base of this arrangement, the EP-B-1621442 with its equivalents in the most important industrialised countries is to be cited. Further improvements of this basic arrangement applied in the present invention are the ones divulged in the Swiss patent Nr. CH/925/07 concerning an “Apparatus with a hydraulic or pneumatic pressure sensor for steering elements”, in the CH/1179/07 concerning a “Method of filling with liquid an elastic tube of a pressure sensor”, in the CH/0867/0 concerning a “Method of adjusting an apparatus comprising a flexible tube”. All these inventions are to be considered as integral parts of the present patent application as they constitute the essential base on which the inventive solution is based.

It thus is the objective of the present invention to offer the driver, preparing to face the dangers of the road, the opportunity of voluntarily undergoing a test that determines aptitude for driving, which depends not only on his blood alcohol level, but on all his psychophysical capacities with respect to tiredness, drug consumption, debilitating medicines taken, the stress level and also to age, etc., i.e. as a whole, his capacity of reacting correctly and within short time spans to external activating solicitations. In particular persons are aimed at which are leaving discos, night clubs, bars, etc., to which the opportunity is to be offered of voluntarily testing their driving capability before they take place behind the wheel. Only after successful completion of the test, which normally will be confirmed on a corresponding printout certificate, or be transmitted directly to his mobile phone, the person will know he is capable to drive more or less without danger. Using a suitable telephone connection the test results can be recalled at any time, e.g. in case of police controls, in order to demonstrate that the driver had undergone and successfully passed the test before taking place behind the wheel. Also as a compulsory test for seniors the application of the reflex measuring apparatus can provide an optimal and reliable demonstration of the ability of driving an automobile, also at driving schools or other competent institutions.

All these objectives are met using the reflex measuring apparatus presenting the characteristics according to the claim 1, in combination, possibly, with the characteristics according to the claims 2 through 10.

The claims 11 and 12 concern two preferred types of application of the inventive reflex measuring apparatus.

The invention will be described in more detail in the following with reference to the illustrations, which show in the

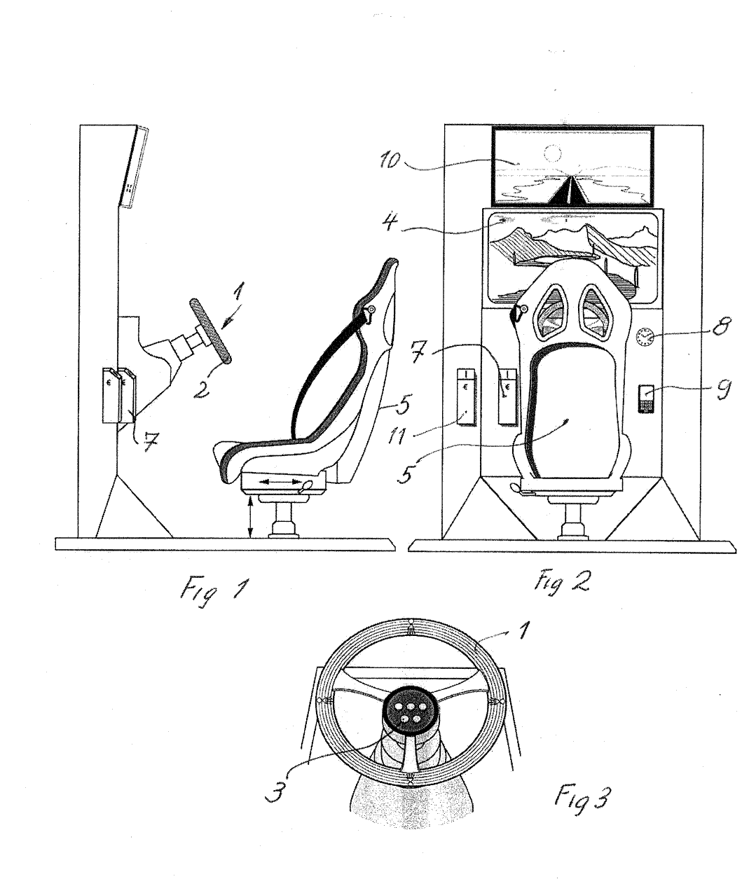

FIG. 1 the inventive reflex measuring apparatus in a lateral view,

FIG. 2 the inventive reflex measuring apparatus seen from the back side,

FIG. 3 a detail of the inventive reflex measuring apparatus showing the steering wheel provided with a device reacting to the variation of pressure.

Before a detailed description of the Figures is given, two general aspects of the present invention are to be considered.

First observation: The inventive reflex measuring apparatus is a new combination of elements all known as such from the state of the art. The individual elements can assume types and shapes that differ from the ones shown in the Figures without exceeding the frame of the present invention. Important, however is the combination of their functions that permits realisation of the objectives aimed at, which can differ also according to the application of the reflex measuring apparatus intended (example: reflex measuring apparatus for discos, reflex measuring apparatus for police controls, driving schools, etc.).

Second observation: Concerning the steering wheel, element always required for measuring the reactivity of the driver, the present invention is based primarily on the abovementioned solution according to the -EB-B-1621442 and its improvements derived from the patent applications cited in the introduction.

In the FIGS. 1 through 3 identical elements are designated using the same reference numbers.

With 1 a steering wheel is designated provided with a device reacting to the variations in the pressure exerted by one or both hands of the driver gripping the rim 2 of the steering wheel 1. The invention is based on the principle that the measure of reactivity of a driver who is driving correctly gripping the steering wheel with one, or preferentially, with both hands can be determined optimally as the time span between the moment of occurrence of the danger signal using a signalling device (which can be a signal of a little lamp 3 or better using a landscape simulator—the appearance of a light, of an unexpected obstacle, e.g. a pedestrian crossing the street, an animal, a hole in the road surface, etc.) and the reaction of the driver. In any case a signalling device 2, 3 is required that can be laid out in various forms, such as e.g. a small lamp 3 mounted onto the hub of steering wheel 1 (compare the FIG. 3) or the screen 4 of a landscape simulator as shown in the FIG. 2. Also acoustic signalling devices (combined or not with the optical ones) can be considered.

For operation of the inventive reflex measuring apparatus also a device, e.g. an electronic device, is required for displaying the reaction times, that can measure the time span between the signal given off by the signalling device 2, 3 and the reaction of the device reacting to the variations in pressure exerted by the hand or hands of the driver, as provided and described in the EP-B-1621442. According to a preferred solution such a device is an analogue or digital chronograph 8 that can measure hundredths or thousandths of a second.

The inventive reflex measuring apparatus furthermore comprises an automobile seat 5 the position of which can be adapted to the physical properties of the driver undergoing the test, and which is provided with a safety belt 6 and a starting device (not shown) of the reflex measuring apparatus, activated when the driver takes place on the seat 5.

This starting device can be laid out in different forms, and experience has shown that a preferred form is obtained in combining the starting device with the fastening device of the safety belt 6 or, in a preferred variant, with a device (not shown) that can detect the presence of a driver on the seat 5, if the seat 5 is provided with such a device (as used in many vehicles for optimally controlling the functions of the air bags).

For certain requirements (e.g. for handing the driver a document proving successful completion of the test, to be presented to police officers in case of blood-alcohol level checks, etc.), the reflex measuring apparatus can comprise also a printer (not shown in the Figure) for printing test data and, for requirements similar to the ones mentioned, a mobile phone connection 9 using which test results can be received automatically or can be recalled from any external phone station, in order to offer proof to the controlling officer, that the driver had successfully passed the test, even if he has no paper printout of the results on hand.

Considering the particular requirements implied by the application of the reflex measuring apparatus, a preferred embodiment of the reflex measuring apparatus provides, that it also comprises a coin pay station 7 for payment of the test by the driver. This solution evidently is laid out in particular for cases of application of the reflex measuring apparatus in discos, bars, night clubs, etc., for preventively documenting, voluntarily of course without constraint, the driving ability of the client leaving the premises. Obviously the operator of the establishment is entitled to adequate indemnity.

It is to be underlined, according to a further preferred embodiment, that the inventive reflex measuring apparatus can represent an optimal instrument adapted to police requirements (e.g. police traffic controls) for determining the driving ability of the driver not only in function of blood-alcohol level, but also in function of tiredness (possibly caused by advanced age of the person, drug consumption, and similar circumstances such as stress) that influence the reactivity of the driver of a vehicle, all recorded on a data storage (SD) acting as a black box, as required in the future by the European authorities.

It thus can be confirmed that the inventive reflex measuring apparatus can be set up in its basic structure as a proper simulator of driving a vehicle on the road, and in its various applications, can bring an important advantage to the road traffic safety and thus can help for considerably reducing the number of accidents—and of victims—caused by persons driving with impaired driving ability, or expressed more simply, unfit to drive.

### LIST OF THE ELEMENTS REFERRED TO IN THE FIGURES

1 steering wheel provided with an apparatus according to the EP-B-1621442
2 rim of the steering wheel
3 little lamp
4 screen
5 seat
6 safety belt
7 coin pay station
8 chronograph
9 mobile phone
10 image projector
11 second coin pay station for soft drinks free of alcohol

1. Reflex measuring apparatus for determining using a test the reactivity of a driver, or respectively his ability of driving a vehicle, comprising the following elements:
a steering wheel (1) of a vehicle provided with a device reacting to the variations in pressure exerted by one or both hands of the driver gripping the rim (2) of the steering wheel (1),
a signalling device (2; 3; 4) that can provoke the reaction of the driver,
a device (8) that can measure the time span between the signal given off by the signalling device (2; 3; 4) and the reaction of the device reacting to the variations in pressure exerted by the hand or the hands of the driver (measure of reactivity of the driver),
a seat (5) of an automobile, the position of which can be adapted, and which is provided with a safety belt (6),
a starting device (6) of the reflex measuring apparatus activated as the driver takes place on the seat (5).

2. Reflex measuring apparatus according to the claim 1,
characterised in that
the signalling device (2; 3) is a little lamp (3) lighting up unexpectedly, requiring the attention of the driver.

3. Reflex measuring apparatus according to the claim 1,
characterised in that
the signalling device (2; 3) comprises a screen (4) of a landscape simulator on which an obstacle appears unexpectedly that requires the attention of the driver.

4. Reflex measuring apparatus according to the claim 1,
characterised in that
the device that can measure the time span is an analogue or digital chronograph (8) that can record hundredths and/or thousandths of s second.

5. Reflex measuring apparatus according to the claim 1,
characterised in that
the starting device is connected to the fastening lock of the safety belt (6).

6. Reflex measuring apparatus according to the claim 1,
characterised in that
the starting device is connected to a device that can detect the presence of a driver on the seat (5).

7. Reflex measuring apparatus according to the claim 1,
characterised in that
it comprises also a printer for printing test data.

8. Reflex measuring apparatus according to the claim 1,
characterised in that
it comprises also a mobile phone connection (9) using which test data can be transmitted or recalled from any external phone station.

9. Reflex measuring apparatus according to the claim 1,
characterised in that
it comprises also a coin pay station (7) using which the test can be paid by the driver.

10. Reflex measuring apparatus according to the claim 1,
characterised in that
it comprises also a projector (10) for transmitting publicity messages and/or video games.

11. Application of the reflex measuring apparatus according to claim 1,
characterised in that
it is specifically adapted to be used in discos, bars, night clubs, driving schools, police stations, etc., for determination the driving ability of the driver/client at the exit of the premises.

12. Application of the reflex measuring apparatus according to claim 1,
characterised in that
it is specifically adapted to be used by police forces for determination of the driving ability of the driver in function not only of the blood alcohol level, but also of the tiredness, drug consumption and similar circumstances that influence the reactivity of a driver of a vehicle.

13. Reflex measuring apparatus according to the claim 1,
characterised in that
it comprises also a soft drink dispenser.
